(19) United States
(12) Patent Application Publication
(10) Pub. No.: US 2024/0009382 A1
(43) Pub. Date: Jan. 11, 2024

(54) DETACHMENT MECHANISM OF A WEARABLE INFUSION DEVICE AND RELATED TECHNOLOGY

(71) Applicant: MEDTRONIC MINIMED, INC., Northridge, CA (US)

(72) Inventors: GianCarlo Seixas, Studio City, CA (US); Matthew William Yavorsky, Granada Hills, CA (US); Lauren N. Knop, Los Angeles, CA (US); Jonathan Scott Yeager, Northridge, CA (US); Rachel Katherine Brockman, Sunnyvale, CA (US)

(21) Appl. No.: 18/349,033

(22) Filed: Jul. 7, 2023

Related U.S. Application Data
(60) Provisional application No. 63/368,004, filed on Jul. 8, 2022.

(51) Int. Cl. A61M 5/142

(52) U.S. Cl. CPC A61M 5/14244; A61M 2005/14268

(57) ABSTRACT

A detachable component of a wearable medical device in accordance with an embodiment of the present technology includes a housing and a latch operably associated with the housing. The latch includes a cam having a first position at which the cam projects outwardly from the housing, a second position at which the cam is shifted inwardly relative to the first position, and a third position at which the cam is shifted inwardly relative to the second position. The cam is resiliently biased from the second position toward the first position. Fully actuating the cam moves the cam from the first position to the second position. The detachable component further includes a handle accessible from outside the housing. The handle is movable in an actuating direction to move the cam from the first or second position to the third position.

### CROSS-REFERENCE TO RELATED APPLICATIONS

This application claims the benefit of U.S. Provisional Application No. 63/368,004, filed Jul. 8, 2022. The disclosure of which are herein incorporated by reference in their entirety.

### TECHNICAL FIELD

This disclosure is related to wearable medical devices, such as wearable infusion devices.

### BACKGROUND

Infusion devices are used to control delivery of medication directly into a patient's bloodstream. In some cases, the delivery of medication from an infusion device is slow and continuous. This can be useful, for example, to maintain a steady concentration of medication in a patient's bloodstream over many hours or days. Infusion devices can also respond quickly (e.g., in near real time) to biometric information from associated sensors. A common use for infusion devices is in the treatment of diabetes. Many patients with diabetes benefit from frequent or continuous infusion of insulin. This need can be temporary, such as during a hospital admission, or long-term. In the latter case, wearable infusion devices can enable a patient to receive necessary insulin infusion therapy while maintaining an active lifestyle. Wearable infusion devices typically include a battery-powered pump, a reservoir, and a cannula. The cannula is maintained in a transcutaneous position at an infusion site, such as under an adhesive patch applied directly to a patient's skin. In some cases, the pump and reservoir are in a separate unit flexibly tethered to the cannula. This unit, for example, can be strapped to a patient or attached to a patient's clothing. Alternatively, the pump and reservoir can be integrated with the cannula into a single unit configured to be directly attached to a patient's skin at an infusion site.

Without modern infusion devices, management of diabetes and other conditions that require continuous or frequent infusion of medication would be far more problematic. For example, many patients suffering from diabetes have difficulty actively monitoring their need for insulin (e.g., by performing blood glucose tests) and responding to this need appropriately (e.g., by self-administering appropriate quantities of insulin). Without assistance, these patients can be subject to a significant, ongoing risk of serious complications including diabetic coma and death. Even when active disease management is within a patient's capabilities, it can be time-consuming and stressful. Infusion devices, therefore, not only save lives, they meaningfully improve the quality of life for patients with diabetes and other diseases. Given that diabetes affects hundreds of millions of people worldwide and is only one example of a disease that often requires continuous or frequent infusion of medication, the importance of infusion devices is difficult to overstate. Accordingly, there is an ongoing need for improvement of these devices. Even small improvements in this field can have major public health benefits.

### SUMMARY

A detachable component of a wearable medical device in accordance with at least some embodiments of the present technology includes a housing, a latch operably associated with the housing, and a handle accessible from outside the housing. The latch includes a cam having a first position at which the cam projects outwardly from the housing and a second position at which the cam is shifted inwardly relative to the first position. The cam also has a third position at which the cam is shifted inwardly relative to the second position. The cam is resiliently biased from the second position toward the first position. In addition, fully actuating the cam moves the cam from the first position to the second position. The handle is movable in an actuating direction to move the cam from the first or second position to the third position.

A wearable medical device in accordance with at least some embodiments of the present technology includes first and second components configured to be detachably connected to one another. The first and second components include first and second housings, respectively. The second component also includes a latch operably associated with the second housing and a handle accessible from outside the second housing. The latch includes a cam having a first position at which the cam projects outwardly from the second housing and a second position at which the cam is shifted inwardly relative to the first position. The cam also has a third position at which the cam is shifted inwardly relative to the second position. The cam is resiliently biased from the second position toward the first position. The first and second components are shaped to constrain relative movement thereof such that detachably connecting the first and second components to one another automatically actuates the cam and thereby moves the cam from the first position to the second position. In addition, the handle is movable in an actuating direction to move the cam from the first or second position to the third position.

A method for operating a wearable medical device in accordance with at least some embodiments of the present technology includes connecting first and second components of the wearable medical device to one another and disconnecting the first and second components from one another. Connecting the first and second components to one another includes sliding a wall of a first housing of the first component against a cam of a latch of the second component. This moves the cam from a first position at which the cam projects outwardly from a second housing of the second component to a second position at which the cam is shifted inwardly relative to the first position. Connecting the first and second components to one another also includes aligning a pocket of the first component at the wall with the cam and thereby enabling the cam to resiliently extend into the pocket. Disconnecting the first and second components from one another includes moving a handle of the second component a first distance in an actuating direction and thereby causing the cam to move out of the pocket to the second position. Disconnecting the first and second components from one another also includes moving the handle a second distance in the actuating direction and thereby causing the cam to move from the second position to a third position at which the cam is shifted inwardly relative to the second position. Moving the handle the second distance in the actuating direction also causes a clasp of the latch to automatically and permanently secure the cam in the third position.

### BRIEF DESCRIPTION OF THE DRAWINGS

Many aspects of the present technology can be better understood with reference to the following drawings. The relative dimensions in the drawings may be to scale with respect to some embodiments of the present technology. With respect to other embodiments, the drawings may not be to scale. The drawings may also be enlarged arbitrarily. For clarity, reference-number labels for analogous components or features may be omitted when the appropriate reference-number labels for such analogous components or features are clear in the context of the specification and all of the drawings considered together. Furthermore, the same reference numbers may be used to identify analogous components or features in multiple described embodiments.

FIG. 1 is a perspective view of a wearable medical device in accordance with at least some embodiments of the present technology.

FIG. 2 is a different perspective view of the device shown in FIG. 1.

FIG. 3 is a front profile view of the device shown in FIG. 1.

FIG. 4 is an exploded perspective view of the device shown in FIG. 1.

FIG. 5 is a perspective view of a first component of the device shown in FIG. 1.

FIG. 6 is a perspective view of a second component of the device shown in FIG. 1.

FIG. 7 is an exploded perspective view of the second component of the device shown in FIG. 1.

FIG. 8 is a front profile view of the second component of the device shown in FIG. 1.

FIG. 9 is a cross-sectional top plan view of the second component of the device shown in FIG. 1 taken along the line A-A in FIG. 8 with a cam of a latch of the second component in a first position.

FIG. 10 is an enlarged view of a portion of FIG. 9.

FIG. 11 is an enlarged cross-sectional top plan view corresponding to FIG. 10 of the second component of the device shown in FIG. 1 taken along the line A-A in FIG. 8 with the cam in a second position.

FIG. 12 is an enlarged cross-sectional top plan view corresponding to FIG. 10 of the second component of the device shown in FIG. 1 taken along the line A-A in FIG. 8 with the cam in a third position.

FIG. 13 is an enlarged cross-sectional top plan view corresponding to FIG. 10 of a second component of a device in accordance with another embodiment of the present technology.

FIG. 14 is a block diagram illustrating a method for operating a wearable medical device in accordance with at least some embodiments of the present technology.

### DETAILED DESCRIPTION

Wearable medical devices and related devices, systems, and methods in accordance with embodiments of the present technology at least partially address one or more problems associated with conventional technologies whether or not such problems are stated herein. Devices in accordance with at least some embodiments of the present technology include innovative features that facilitate attaching components of the devices to one another and detaching the components from one another as needed. For example, the attaching and detaching processes can be simple and intuitive to facilitate performance of these operations by patients with little or no instructional guidance. Furthermore, when attached, the connection between the components can be secure enough to prevent the components from detaching from one another during use, such as during exercise or other activities that may subject the device to unpredictable forces.

Features that facilitate attaching and detaching components of wearable medical devices can be especially useful in the context of wearable infusion devices, such as those used to deliver insulin solution directly into a patient's bloodstream. In these and certain other devices, it can be useful for some elements to be more durable than other elements. For example, it can be useful for some elements to be multi-use and for other elements to be single-use (e.g., disposable). As a particular example, it is generally recommend to use a new cannula with every new infusion site to reduce the risk of infection. Moreover, the reservoir of infusion liquid in an infusion device is sometimes nonrefillable and, therefore, only usable for a limited period of time before it becomes depleted. In contrast, the pump motor, power source, wireless receiver, and other electronics are typically more compatible with reuse. These elements also tend to be more expensive than the cannula and the reservoir, such that replacing them every time a new infusion site is created would likely be cost prohibitive. Accordingly, it can be useful to incorporate the cannula, reservoir, and/or other elements most compatible with single-use into one component of an infusion device and to incorporate the pump motor, power source, wireless communication elements, other electronics, and/or other elements most compatible with multi-use into another component of the infusion device. This division can also facilitate protecting liquid-sensitive elements from exposure to infusion liquid. For these and/or other reasons, an intuitive mechanism for attaching and detaching components of infusion devices has the potential to reduce costs and improve usability, thereby expanding access to such devices for long-term use.

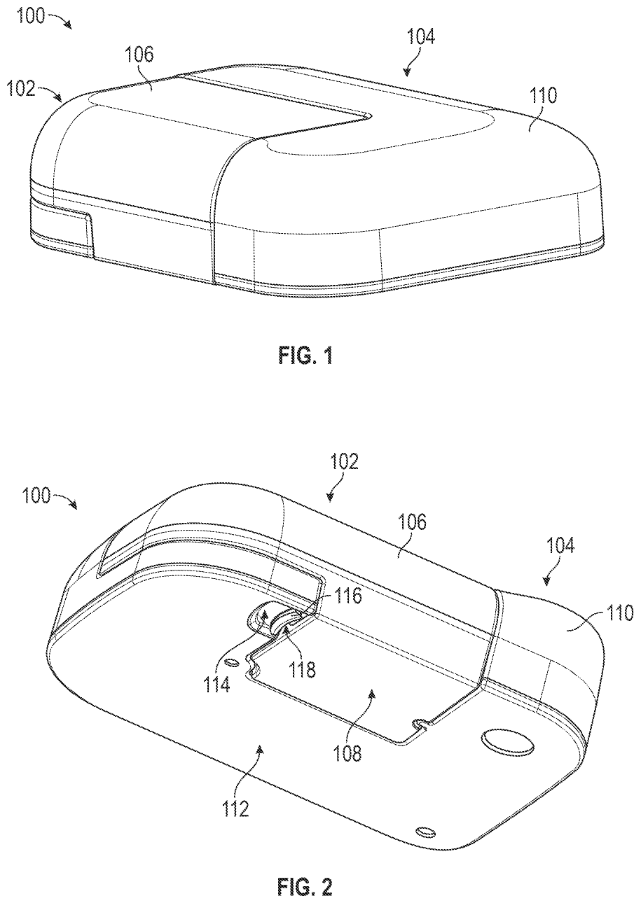

Specific details of several embodiments of the present technology are disclosed herein with reference to FIGS. 1-14. It should be noted, in general, that other embodiments in addition to those disclosed herein are within the scope of the present technology. For example, embodiments of the present technology can have different configurations, components, and/or operations than those disclosed herein. Moreover, a person of ordinary skill in the art will understand that embodiments of the present technology can have configurations, components, and/or operations in addition to those disclosed herein and that these and other embodiments can be without configurations, components, and/or operations disclosed herein without deviating from the present technology.

FIGS. 1 and 2 are different perspective views of a wearable medical device 100 in accordance with at least some embodiments of the present technology. With reference to FIGS. 1 and 2 together, the wearable medical device 100 can include a first component 102 and a second component 104 detachably connected to one another. In the depicted embodiment, the first component 102 is a reusable component and the second component 104 is a disposable component. In other embodiments, the first component 102 may be the disposable component and the second component 104 may be the reusable component. The first component 102 can include a first housing 106 having a first contact surface 108 through which the first housing 106 is configured to be connected to skin of a patient while the patient wears the wearable medical device 100. Similarly, the second component 104 can include a second housing 110 having a second contact surface 112 through which the second housing 110 is configured to be connected to the patient's skin while the patient wears the wearable medical device 100. As shown in FIG. 2, the first and second contact surfaces 108, 112 can be in the same plane when the first and second components 102, 104 are detachably connected to one another. For example, the first and second contact surfaces 108, 112 can be positioned to both engage a single adhesive patch (not shown) when the first and second components 102, 104 are detachably connected to one another.

As also shown in FIG. 2, the second housing 110 can include a window 114 at the second contact surface 112. The second component 104 can include a handle 116 accessible from outside the second housing 110 via the window 114. As discussed below, the handle 116 is operable to manually disengage the first and second components 102, 104 from one another and thereby enable the first and second components 102, 104 to be detached from one another. Positioning of the window 114 and of the handle 116 at the second contact surface 112 can be useful, for example, to reduce the likelihood of unintentionally actuating the handle 116 while the wearable medical device 100 is in use. For example, until a patient removes the wearable medical device 100 from their skin, the handle 116 is inaccessible. To avoid interfering with a stable connection between the wearable medical device 100 and a patient's skin or for other reasons, the handle 116 can be flush with or recessed relative to the first and second contact surfaces 108, 112. In these and other cases, the handle 116 can define a recess 118 configured to receive a fingertip when the first and second components 102, 104 are detachably connected to one another. Accordingly, a user of the wearable medical device 100 can actuate the handle 116 by positioning a fingertip in the recess 118 and using the fingertip to slide the handle 116 in an actuating direction from one side of the window 114 to an opposite side of the window 114.

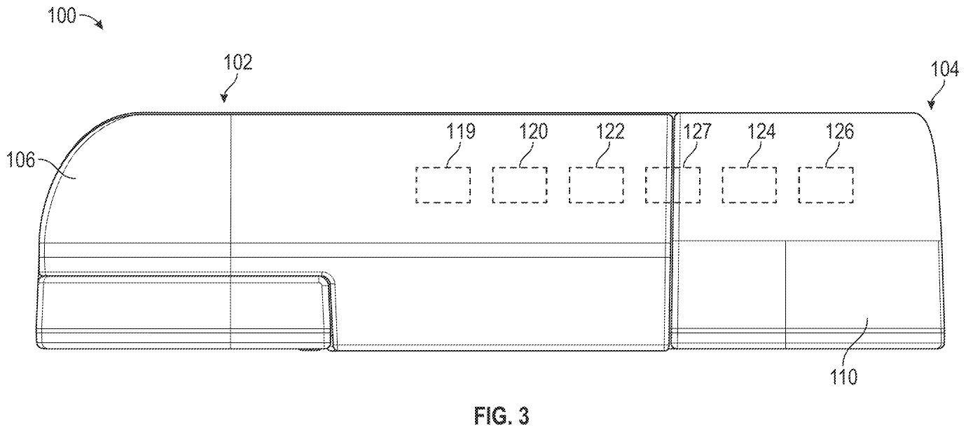

FIG. 3 is a front profile view of the device shown in FIG. 1. As shown in FIG. 3, the wearable medical device 100 can include a pump motor 119, a wireless receiver 120, and a controller 122 at the first component 102 within first housing 106. The wearable medical device 100 can further include a reservoir 124 and a cannula 126 at the second component 104 within second housing 110. The reservoir 124 can be configured to hold a quantity of liquid (e.g., insulin solution) to be delivered to the patient's bloodstream via the cannula 126. The cannula can be manually or otherwise extendable from a retracted position within the second housing 110 (as indicated in FIG. 3) toward a transcutaneous extended position. In some cases, the reservoir 124 is refillable. In other cases, the reservoir 124 is not refillable. The controller 122 can be configured to cause liquid from the reservoir 124 to flow to the cannula 126 at least partially in response to a signal received by the wireless receiver 120. For example, the wireless receiver 120 can receive a signal indicating that a patient's blood-glucose is rising. Based on this signal alone or in combination with other information, the controller 122 can instruct the pump motor 119 to move insulin solution from the reservoir 124 into the patient's bloodstream via the cannula 126. The signal can be based on biometric data, user input, and/or other information from an external device in wired or wireless communication with the wearable medical device 100. The external device, for example, can be a sensor, such as a constant glucose monitor, or a computer, such as smart phone with data input by a user. Alternatively or in addition, the wearable medical device 100 can include an internal sensor, such as an internal glucose sensor. Glucose and other sensors often have limited operational lives. Accordingly, it can be useful to incorporate an internal sensor into the second component 104 rather than the first component 102. In other cases, an internal sensor can be relatively durable and most suitably incorporated into the first component 104 rather than the second component 104.

In at least some embodiments, the wearable medical device 100 includes a slidingly detachable interface 127 between the first and second components 102, 104. The interface 127 can be electrical, fluidic, and/or mechanical. For example, when the pump is within the second component 104 and its power source is within the first component 102 and in other cases, the interface 127 can include electrodes (not shown) at the first and second components 102, 104, respectively. The electrodes can slide into electrical contact as the first and second components 102, 104 are detachably connected to one another. While in electrical contact with one another, the electrodes can enable power from the power source to flow to the pump. In the illustrated embodiment, the first component 102 is the reusable component and the second component 104 is the single use, disposable component. This can enable the pump motor 119, the wireless receiver 120, and the controller 122 to be more sophisticated and costly than would otherwise be practical, protect these elements from liquid within the second component 104, and/or have other benefits. Further, by having two components, less waste is discarded, thus providing an environmental benefit as well. In other embodiments, these elements, the reservoir 124, and the cannula 126 can have other suitable arrangements. Moreover, the first and second components 102, 104 can have other levels of durability. For example, both the first and second components 102, 104 can be single-use or multi-use.

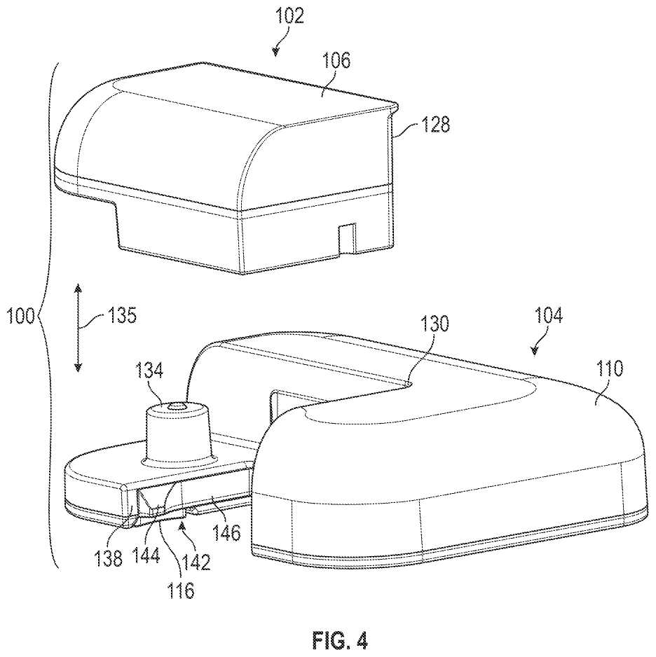

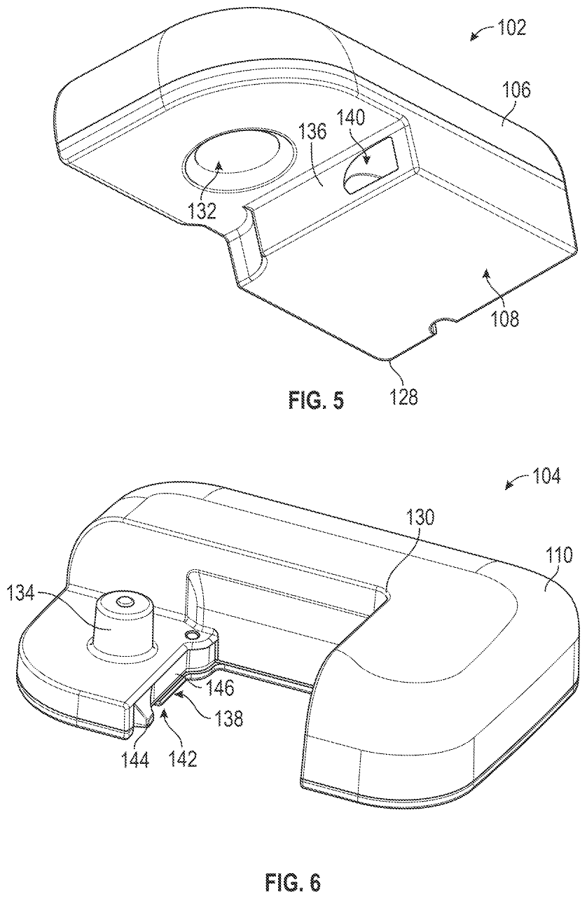

FIG. 4 is an exploded perspective view of the wearable medical device 100. FIGS. 5 and 6 are perspective views of the first and second components 102, 104, respectively. With reference to FIG. 4-6 together, the first and second components 102, 104 can be shaped to constrain their relative movement during the attaching process. For example, the first housing 106 can include walls that meet at an outside corner 128. The second housing 110 can include walls that meet at an inside corner 130. Aligning the outside and inside corners 128, 130 can dictate relative positions of the first and second components 102, 104 in two dimensions, making relative movement in the remaining dimension during the connecting process intuitive for a user. Furthermore, the first and second components 102, 104 can include a mating socket 132 and pillar 134, respectively. Together with apposition of the walls at the inside and outside corners 126, 128, alignment of the socket 132 and the pillar 134 can constrain relative movement of the first and second components 102, 104 to a connecting/disconnecting axis (indicated by a double arrow 135 in FIG. 4) during the connecting process.

The first and second components 102, 104 can include a first wall 136 and a second wall 138, respectively, configured to be parallel and adjacent when the first and second components 102, 104 are detachably connected to one another. As shown in FIG. 5, the first component 102 can include a pocket 140 at the first wall 136. As shown in FIG. 6, the second component 104 can include a latch 142 operably associated with the second housing 110. The latch 142 can include a cam 144 and an arm 146 through which the cam 144 is connected to other portions of the second component 104. In FIG. 6, the cam 144 is shown in a first position in which the cam 144 projects outwardly from the second housing 110. For example, the cam 144 in the first position can project outwardly from a plane defined by an outer surface of the second wall 138. In the illustrated embodiment, an outer surface of the arm 146 is in substantially the same plane as the outer surface of the second wall 138 when the cam 144 is in the first position. In other embodiments, the arm 146 can be within the second housing 110 and behind the second wall 138. In still other embodiments, the arm 146 can be omitted and the cam 144 can be connected to other portions of the second component 104 in another suitable manner.

With reference again to FIGS. 4-6, when relative movement of the first and second components 102, 104 is constrained to the connecting/disconnecting axis, the first and second walls 136, 138 are parallel or substantially parallel to one another. Accordingly, relative movement of the first and second components 102, 104 during the connecting process can cause the first wall 136 to contact and automatically actuate the cam 144. For example, a portion of the first wall 136 below the pocket 140 can slidingly engage a sloped upper portion of the cam 144 and thereby cause the cam 144 to resiliently retract into the second housing 110. As the relative movement continues, the pocket 140 will become aligned with the cam 144, enabling the cam 144 to resiliently extend into the pocket 140. With the cam 144 at least partially received within the pocket 140, a relatively flat lower portion of the cam 144 can interact with the portion of the first wall 136 below the pocket 140 to prevent the first and second components 102, 104 from detaching from one another.

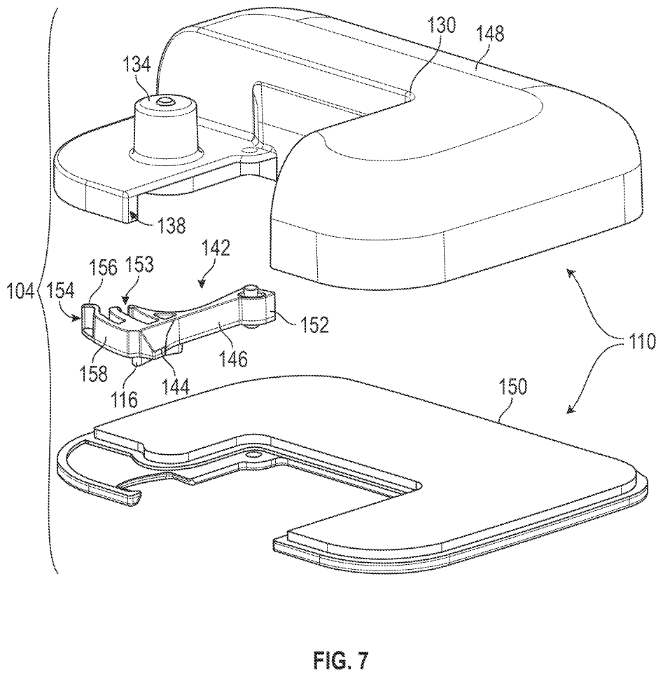

FIG. 7 is an exploded perspective view of the second component 104. As shown in FIG. 7, the second component 104 can include a shell 148 and a plate 150 connectable to one another to form the second housing 110. The latch 142 can include a connector 152 at which the latch 142 is fixedly connected to the second housing 110. For example, the connector 152 can be at one end of the arm 146 and the cam 144 can be at an opposite end of the arm 146. In these and other cases, the arm 146 can be flexible and can resiliently connect the cam 144 to the second housing 110. Suitable materials for the arm 146 include plastic and metal. For example, the arm 146 and the structures carried by the arm 146 can be molded together out of only plastic. Alternatively, the arm 146 can include a thin metal sheet to which other portions of the arm 146 and the structures carried by the arm 146 are overmolded. Incorporating metal into the arm 146 can be useful, for example, to increase the resiliency of the arm 146, such as by reducing or eliminating polymer creep. As shown in FIG. 7, the handle 116 can be carried by the latch 142 such that moving one automatically moves the other. Thus, the cam 144 can be configured to move relative to the second housing 110 in response to being actuated and/or in response to movement of the handle 116. As also shown in FIG. 7, the latch 142 can further include a slot 153. The latch 142 can also include a clasp 154 having a hook 156 and a flexible stem 158 through which the hook 156 is resiliently connected to other portions of the latch 142 and to the handle 116. As discussed in detail below, slot 153 and the clasp 154 can interact with other structures of the second component 104 to facilitate detaching the first and second components 102, 104 from one another.

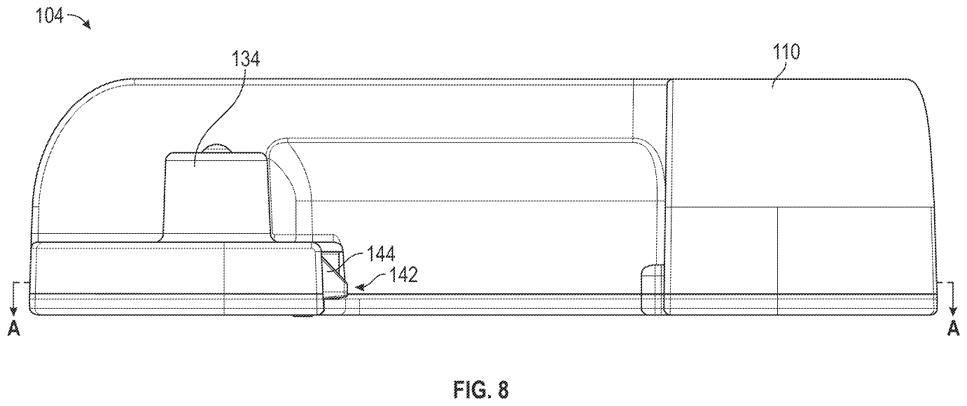

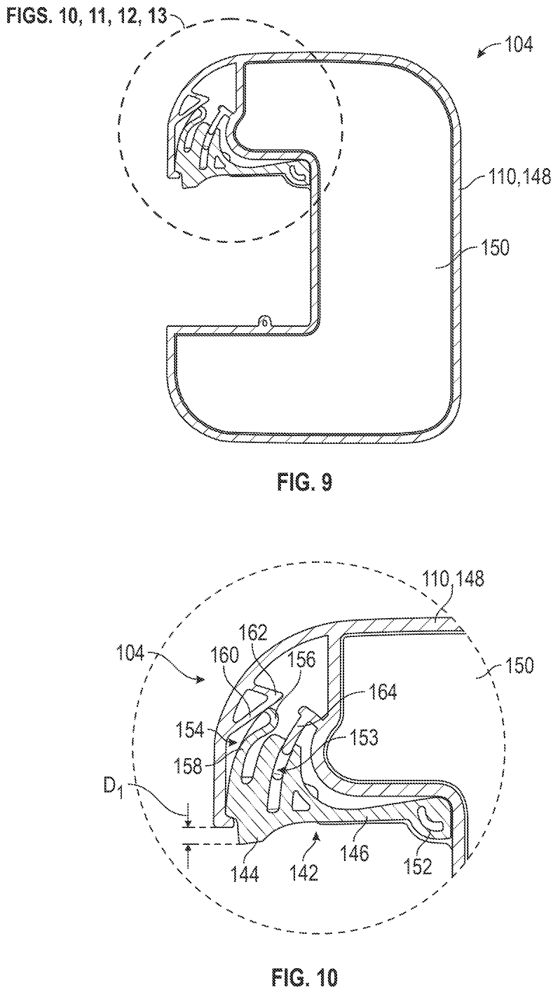

FIG. 8 is a front profile view of the second component 104. FIG. 9 is a cross-sectional top plan view of the second component 104 taken along the line A-A in FIG. 8 with the cam 144 in the first position. FIG. 10 is an enlarged view of a portion of FIG. 9. With reference to FIGS. 8-10 together, the second component 104 includes a ramp 160 and a catch 162 at one end of the ramp 160. The second component 104 can further include a finger 164 configured to be at least partially received within the slot 153. The ramp 160, the catch 162, and the finger 164 can be carried by the second housing 110. For example, these structures can be integrally formed with walls of the second housing 110 or otherwise fixedly connected to the second housing 110. During a process of disconnecting the first and second components 102, 104 from one another, the finger 164 can interact with the slot 153. Likewise, the ramp 160 and the catch 162 can interact with the clasp 154. These interactions are further discussed below with reference to FIGS. 10-12.

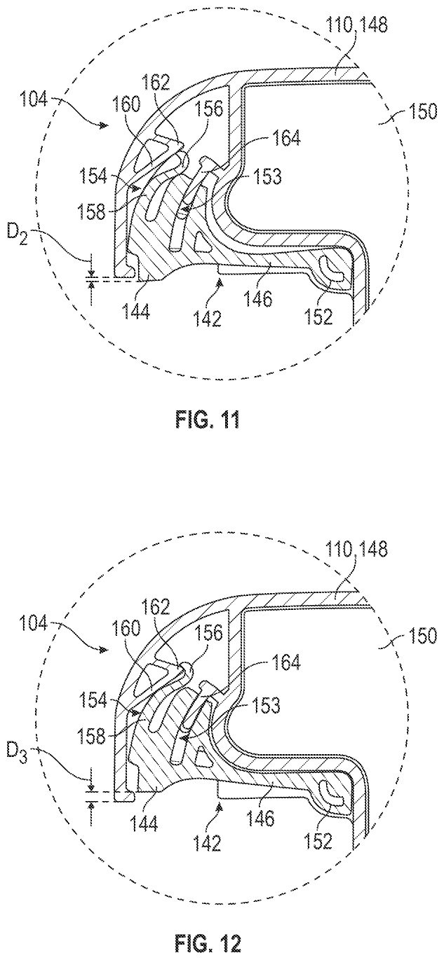

FIGS. 11 and 12 are enlarged cross-sectional top plan views corresponding to FIG. 10 but showing the cam 144 in a second position and a third position, respectively. With reference to FIGS. 10-12 together, the cam 144 can be progressively more retracted (i.e., inwardly shifted) from the first position to the second position and then to the third position. For example, in the first position (FIG. 10), the cam 144 can project a distance D₁ from a plane of the outer surface of the second wall 138. In the second position (FIG. 11), the cam 144 can project a smaller distance D₂ from the plane. Finally, in the third position (FIG. 12), the cam 144 can be inwardly spaced apart a distance D₃ from the plane and latched into the third position. The arm 146 can resiliently bias the cam 144 from the second position toward the first position and from the third position toward the second position.

In at least some cases, the second position is a fully actuated position of the cam 144. For example, the second position can correspond to a maximum automatic retraction of the cam 144 in response to sliding interaction with the first wall 136 (FIG. 5) while connecting the first and second components 102, 104 to one another. Connecting the first and second components 102, 104 to one another can fully actuate the cam 144 before the cam 144 aligns with the pocket 140 (FIG. 5). Once the cam 144 aligns with the pocket 140, a resilient bias on the cam 144 can cause it to extend into the pocket 140 and into a fully engaged position. When fully extended into the pocket 140, the cam 144 can be in the first position or in another position between the first position and the second position. The distance D₂ can correspond to a minimum spacing between first and second walls 136, 138 while connecting the first and second components 102, 104 to one another. In the illustrated embodiment, the spacing is nonnegligible. In other embodiments, the spacing can be negligible. For example, the first and second walls 136, 138 can be in slidingly contact while connecting the first and second components 102, 104 to one another. In these and other cases, an outermost surface of the cam 144 can be substantially flush with the outer surface of the second wall 138 when the cam 144 is in the second position.

With reference to FIGS. 2 and 10-12 together, the handle 116 can be movable in the actuating direction to move the cam 144 from the first or second position to the third position. Thus, the handle 116 can be used to hyper-retract the cam 144. In at least some cases, a first distance the cam 144 moves from the first position to the second position and a second distance the cam 144 moves from the second position to the third position can be similar. For example, these distances can differ by less than 25% or by less than 50%. As the cam 144 moves from the first position toward the third position, the finger 164 can move progressively further into the slot 153. Interaction between the finger 164 and the slot 153 can help to guide movement of the latch 142. The clasp 154 can be configured to automatically secure the cam 144 in the third position in response to moving the handle 116 in the actuating direction to move the cam 144 from the first or second position to the third position. This can be useful, for example, to make detaching the first and second components 102, 104 from one another more intuitive and easier. For example, it can be more intuitive and easier for a user to release the handle 116 before separating the first and second components 102, 104 from one another rather than having to hold the handle 116 in an actuated position while separating the first and second components 102, 104 from one another.

Moving the handle 116 in the actuating direction can cause the hook 156 to slide against the ramp 160 and thereby increase tension on the stem 158. During the movement corresponding to movement of the cam 144 from the second position to the third position, the hook 156 can eventually move past the ramp 160, thereby releasing at least some of the stored tension on the stem 158. Releasing this tension can cause the hook 156 to resiliently engage the catch 162. Once the hook 156 engages the catch 162, the user does not have to continue to hold the latch 142 in the retracted position to disengage the first and second components 102, 104. In some cases, securing the cam 144 in the third position is permanent. For example, the clasp 154, the ramp 160, and the catch 162 can be sealed within the second housing 110 so as to be inaccessible to an end user. Making the process permanent can, for example when the latch 142 is on the disposable component, make it impossible for a user to accidently reattach an empty, used component back to the reusable component. In other cases, securing the cam 144 in the third position can be reversible. Making the process reversable may be advantageous if the latch 142 is located on the reusable component.

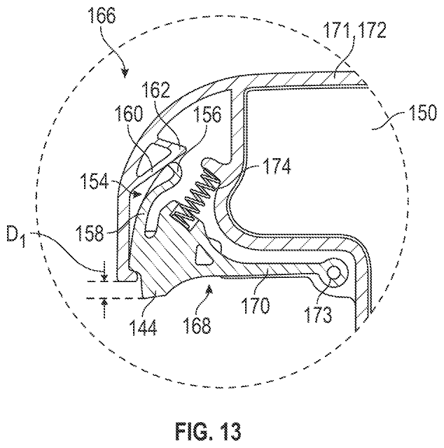

FIG. 13 is an enlarged cross-sectional top plan view corresponding to FIG. 10 of a second component 166 in accordance with another embodiment of the present technology. As shown in FIG. 13, the second component 166 can include a latch 168 including an arm 170 through which the cam 144 is rotatably connected to a shell 171 of a second housing 172. The arm 170 can be rotatably connected to the shell 171 at a connector 173. The second component 166 can further include a spring 174 between the cam 144 and the second housing 172. The spring 174 can bias the cam 144 from the second position toward the first position and from the third position toward the second position. With reference to FIGS. 10 and 13 together, potential advantages of the spring 174 over the resilient arm 146 include greater force and lower stress on the connection point (i.e., connector 152 in the previous example). Potential advantages of the resilient arm 146 over the spring 174 include ease of manufacture, cost, and reliability. Other systems and structures for biasing the cam 144 toward the first position are also possible.

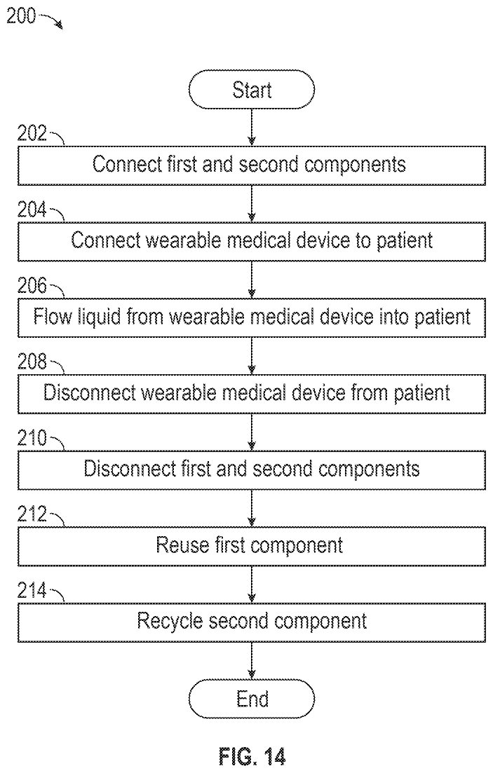

FIG. 14 is a block diagram illustrating a method 200 for operating a wearable medical device in accordance with at least some embodiments of the present technology. For simplicity, aspects of the method 200 will be further described primarily in the context of the wearable medical device 100 described herein. It should be understood, however, that the method 200, when suitable, and/or portions of the method 200, when suitable, can be practiced with respect to other devices in accordance with embodiments of the present technology.

With reference to FIGS. 1-14 together, the method 200 includes connecting the first and second components 102, 104 to one another (block 202). This can include sliding the first wall 136 against the cam 144 to move the cam 144 from the first position (FIG. 10) to the second position (FIG. 11) and then aligning the pocket 140 with the cam 144 and thereby enabling the cam 144 to resiliently extend into the pocket 140. Next, the method 200 includes connecting the wearable medical device 100 to a patient (block 204). In at least some cases, this includes adhesively connecting the wearable medical device 100 to the patient's skin via the first and second contact surfaces 108, 112. In these and other cases, connecting the wearable medical device 100 to the patient can cause the handle 116 to become inaccessible. This can be useful, for example, to prevent the handle 116, and thus the latch 142, from being actuated while the wearable medical device 100 is in use. After connecting the wearable medical device 100 to the patient, the method 200 includes flowing liquid (e.g., insulin solution) from the reservoir 124 into the patient's bloodstream via the cannula 126 (block 206). This can be at least partially in response to a signal received at the wireless receiver 120, at least partially in response to an input from the patient, and/or based on one or more other factors.

The method 200 further includes disconnecting the wearable medical device 100 from the patient (block 208). For example, an adhesive connection between the wearable medical device 100 and the patient's skin can be broken. Disconnecting the wearable medical device 100 from the patient can expose the handle 116. Next, the method 200 includes disconnecting the first and second components 102, 104 from one another (block 210). This can include moving the handle 116 a first distance in the actuating direction and thereby causing the cam 144 to move out of the pocket 140 to the second position (FIG. 11) and, in a continuation of the same motion, moving the handle 116 a second distance in the actuating direction and thereby causing the cam 144 to move to the third position (FIG. 12). Moving the handle 116 the second distance in the actuating direction can also cause the clasp 154 to automatically and permanently secure the cam 144 in the third position (FIG. 12). Without interference from the cam 144, the first and second components 102, 104 can then be moved away from one another along the connecting/disconnecting axis. After the first and second components 102, 104 are disconnected from one another, the method 200 includes reusing the first component (block 212) and recycling the second component (block 214).

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may be disclosed herein in the context of those embodiments, other embodiments may also exhibit such advantages, and not all embodiments need necessarily exhibit such advantages or other advantages disclosed herein to fall within the scope of the present technology. This disclosure and the associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” “including,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various structures. It should be understood that such terms do not denote absolute orientation. Furthermore, reference herein to “one embodiment,” “an embodiment,” or similar phrases means that a particular feature, structure, operation, or characteristic described in connection with such phrases can be included in at least one embodiment of the present technology. Thus, such phrases as used herein are not necessarily all referring to the same embodiment. Finally, it should be noted that various particular features, structures, operations, and characteristics of the embodiments described herein may be combined in any suitable manner in additional embodiments in accordance with the present technology.

1. A detachable component of a wearable medical device, the component comprising:
a housing;
a latch operably associated with the housing, wherein the latch includes a cam, and
wherein:
the cam has a first position at which the cam projects outwardly from the housing,
the cam has a second position at which the cam is shifted inwardly relative to the first position,
the cam has a third position at which the cam is shifted inwardly relative to the second position,
the cam is resiliently biased from the second position toward the first position, and
fully actuating the cam moves the cam from the first position to the second position; and
a handle accessible from outside the housing, wherein the handle is movable in an actuating direction to move the cam from the first or second position to the third position.

2. The detachable component of claim 1, wherein:
fully actuating the cam moves the cam a first distance from the first position to the second position;
the handle is movable in the actuating direction to move the cam a second distance from the second position to the third position; and
the first and second distances differ by less than 50%.

3. The detachable component of claim 1, wherein:
the latch includes an arm through which the cam is resiliently connected to the housing; and
the arm biases the cam from the first position toward the second position.

4. The detachable component of claim 1, wherein:
the housing includes a wall having an outer surface defining a plane;
the cam in the first position projects outwardly from the plane; and
the cam in the third position is inwardly spaced apart from the plane.

5. The detachable component of claim 1, the latch includes an arm through which the cam is rotatably connected to the housing.

6. The detachable component of claim 5, further comprising a spring between the cam and the housing, wherein the spring biases the cam from the second position toward the first position.

7. The detachable component of claim 1, wherein:
the housing includes:
a contact surface through which the housing is configured to be connected to skin of a patient while the patient wears the medical device, and
a window at the contact surface; and
the handle is accessible from outside the housing via the window.

8. The detachable component of claim 7, wherein the handle is flush with or recessed relative to the contact surface.

9. The detachable component of claim 1, wherein the latch includes a clasp configured to automatically secure the cam in the third position in response to moving the handle in the actuating direction to move the cam from the first or second position to the third position.

10. The detachable component of claim 9, wherein the clasp is configured to permanently secure the cam in the third position in response to moving the handle in the actuating direction to move the cam from the first or second position to the third position.

11. The detachable component of claim 9, further comprising a catch carried by the housing wherein:
the clasp includes:
a hook, and
a stem through which the hook is resiliently connected to the handle; and
moving the handle in the actuating direction to move the cam from the first or second position to the third position causes the hook to resiliently engage the catch.

12. The detachable component of claim 11, further comprising a ramp carried by the housing, wherein:
moving the handle in the actuating direction causes the hook to slide against the ramp and thereby increase tension on the stem;
moving the handle in the actuating direction to move the cam from the first or second position to the third position causes the hook to move past the ramp; and
moving the hook past the ramp releases tension on the stem and causes the hook to resiliently engage the catch.

13. A wearable medical device, comprising:
a first component including a first housing; and
a second component configured to be detachably connected to the first component,
wherein the second component includes:
a second housing,
a latch operably associated with the second housing, and
a handle accessible from outside the second housing, and
wherein:
the latch includes a cam having:
a first position at which the cam projects outwardly from the second housing,
a second position at which the cam is shifted inwardly relative to the first position, and
a third position at which the cam is shifted inwardly relative to the second position,
the cam is resiliently biased from the second position toward the first position,
the first and second components are shaped to constrain relative movement thereof such that detachably connecting the first and second components to one another automatically actuates the cam and thereby moves the cam from the first position to the second position, and
the handle is movable in an actuating direction to move the cam from the first or second position to the third position.

14. The wearable medical device of claim 13, further comprising:
a wireless receiver at the first component;
a reservoir at the second component;
a cannula at the second component; and
a controller configured to cause liquid from the reservoir to flow to the cannula at least partially in response to a signal received by the wireless receiver.

15. The wearable medical device of claim 13, wherein:
the first component includes a pocket; and
the cam is at least partially received within the pocket when the first and second components are detachably connected to one another.

16. The wearable medical device of claim 13, wherein:
the first component is multi-use; and
the second component is single-use.

17. The wearable medical device of claim 13, wherein:
one of the first and second components includes a pillar;
the other of the first and second components includes a socket; and
alignment of the pillar and the socket constrains relative movement of the first and second components while detachably connecting the first and second components to one another.

18. The wearable medical device of claim 13, wherein:
the first housing includes a first wall;
the second housing includes a second wall;
the cam projects outwardly from the second wall when the cam is in the first position; and
the first and second walls are parallel and adjacent when the first and second components are detachably connected to one another.

19. The wearable medical device of claim 18, wherein the handle defines a recess configured to receive a fingertip when the first and second components are detachably connected to one another.

20. The wearable medical device of claim 13, wherein:
the first housing includes a first contact surface through which the first housing is configured to be connected to skin of a patient while the patient wears the medical device;
the second housing includes:
a second contact surface through which the second housing is configured to be connected to the skin while the patient wears the medical device, and
a window at the second contact surface; and
the handle is accessible from outside the second housing via the window.

21-25. (canceled)
